SEQUENCE LISTING (2 sequences)

SEQ ID NO: 1 - protein, 34 aa, synthetic construct
IKPEAPGEDASPEELVRYYAGLRHWLNLVTRQRY

SEQ ID NO: 2 - protein, 34 aa, synthetic construct
  VAR_SEQ 9: note = aspartic acid, glycine, glutamic acid, or asparagine
  VAR_SEQ 16: note = valine, aspartic acid, glutamine, or histidine
  VAR_SEQ 17: note = arginine or lysine
  VAR_SEQ 21: note = serine, threonine, alanine, glycine, or methionine
  VAR_SEQ 25: note = tyrosine, tryptophan, or phenylalanine
  VAR_SEQ 28: note = leucine, alanine, isoleucine, valine, or methionine
IKPEAPGEXASPEELXXYYAXLRHXLNXVTRQRY

(19) United States
(12) Patent Application Publication
(10) Pub. No.: US 2026/0109743 A1
(43) Pub. Date: Apr. 23, 2026

(54) PYY3-36 Analog and Use Thereof in Treating Disease

(71) Applicant: Tao Sun, Xiamen (CN)

(72) Inventor: Tao Sun, Xiamen (CN)

(21) Appl. No.: 18/982,094

(22) Filed: Dec. 16, 2024

(30) Foreign Application Priority Data
Oct. 18, 2024 (CN) 202411463164.8

(51) Int. Cl. C07K 14/47; A61K 38/00; A61P 1/16; A61P 3/04; A61P 3/10; A61P 25/22; A61P 25/24

(52) U.S. Cl. CPC C07K 14/4703; A61P 1/16; A61P 3/04; A61P 3/10; A61P 25/22; A61P 25/24; A61K 38/00

(57) ABSTRACT

The present disclosure relates to the field of medicine and discloses a novel human PYY₃₋₃₆ analog. Compared with the naturally-occurring sequence of PYY₃₋₃₆, the analog has a remarkable effect on ameliorating metabolic diseases such as obesity, diabetes and fatty liver, and on mood disorders such as anxiety and depression.

### CROSS-REFERENCE TO RELATED APPLICATION

This application claims the benefit of, and priority to Chinese Patent Application No. 202411463164.8, filed Oct. 18, 2024, the contents of which are herein incorporated by reference in their entirety and for all purposes.

### INCORPORATION BY REFERENCE

This application includes a sequence listing in eXtensible Markup Language (a “xml” file) that is submitted herewith named 2408831.xml created on Dec. 6, 2024, and 5,173 bytes in size. This sequence listing is incorporated by reference herein.

### BACKGROUND

### Technical Field

The present disclosure relates to the technical field of biomedicine, in particular to a PYY₃₋₃₆ analog and use thereof in treating a disease.

### Technical Considerations

Peptide tyrosine tyrosine (PYY) is a 36-amino acid peptide, which is mainly released by L cells in the distal gastrointestinal tract, ileum and colon^{[1]}. PYY belongs to the neuropeptide Y (NPY) family and can widely bind with neuropeptide Y Receptor (NPY Receptor, NPYR). PYY has two endogenous forms, PYY₁₋₃₆ and PYY₃₋₃₆. PYY₃₋₃₆ is produced by the cleavage of the enzyme dipeptidyl peptidase IV (DPP-IV) and has the strongest binding ability to NPY2R^{[1]}. Due to mediation by NPYR^{[2]}, PYY₃₋₃₆ plays a role in suppressing appetite and promoting insulin secretion, is able to inhibit gastrointestinal peristalsis and pancreatic secretion, to regulate intestinal permeability and inflammation in many parts, and to improve diseases related to metabolism, cardiovascular, nervous, digestive and other systems^{[3, 4]}. In view of the wide expression of PYY receptor NPYR in the body, PYY₃₋₃₆ has attracted much attention in the study of diseases such as obesity, diabetes, musculoskeletal diseases, and cancer.

Obesity and its accompanying diseases have become globally important health problems^{[5]}, for example, gallbladder cancer, renal cancer, thyroid cancer, leukemia, liver cancer and ovarian cancer are all related to obesity^{[6]}. Obesity may cause chronic diseases such as diabetes mellitus (DM), fatty liver (FL), cardiovascular diseases, and mental disorders^{[7-9]}. The World Health Organization (WHO) defines obesity as “abnormal or excessive fat accumulation that may damage health”^{[10]}. Excessive alcohol drinking, insufficient exercise and binge eating may cause excessive accumulation of body fat and overweight^{[11]}. DM is a chronic metabolic disease, which is characterized by insufficient insulin secretion by islet β cells and increased blood glucose caused by insulin resistance in tissues. Obesity, sedentary, high-calorie diet, and rising aging population are the main driving factors of DM. FL is a disease of pathological change in liver. When liver cells contain more than 5% of triglycerides, FL is developed^{[12]}. Excessive alcohol drinking and high-calorie food intake are the main causes of chronic liver disease^{[13]}.

PYY₃₋₃₆ is easily degraded in the body and is not suitable for the usage of treatment. At present, long-acting PYY₃₋₃₆ analogs (NN9748 and NNC0165-1875) are in phase I clinical trials for the treatment of obesity, and combined application of NNC0165-1875 and semaglutide is in phase II clinical research.

### SUMMARY

The present disclosure provides a novel PYY₃₋₃₆ analog, which is derived from a human PYY₃₋₃₆ sequence fragment with a mutation at a specific site. Compared with natural PYY₃₋₃₆ sequence, it can play a more effective role in the body, including but not limited to ameliorating conditions such as obesity, diabetes and fatty liver. In addition, it can significantly ameliorate symptoms of mood disorders such as anxiety and depression.

For the above purpose, a first non-limiting aspect of the present disclosure provides a PYY₃₋₃₆ analog comprising an amino acid sequence represented by Formula I below:

Formula I
(SEQ ID NO: 2)
IKP EAP GEX₁₁ ASP EEL X₁₈X₁₉Y YAX₂₃ LRH X₂₇LN
X₃₀VT RQRY.

Without changing the secondary structure of Formula I, amino acid substitution at a specific site is realized according to the principle of conservative substitution of amino acids, wherein:
X₁₁ is selected from the group consisting of aspartic acid, glycine, glutamic acid, and asparagine;
X₁₈ is selected from the group consisting of valine, aspartic acid, glutamine, and histidine;
X₁₉ is selected from the group consisting of arginine and lysine;
X₂₃ is selected from the group consisting of serine, threonine, alanine, glycine, and methionine;
X₂₇ is selected from the group consisting of tyrosine, tryptophan, and phenylalanine; and
X₃₀ is selected from the group consisting of leucine, alanine, isoleucine, valine, and methionine.

In some non-limiting embodiments, the PYY₃₋₃₆ analog comprises a polypeptide sequence represented by Formula II:

Formula II
(SEQ ID NO: 1)
IKP EAP GED ASP EEL VRY YAG LRH WLN LVT RQRY.

A second non-limiting aspect of the present disclosure provides a composition comprising a PYY₃₋₃₆ analog of Formula I or II, and a pharmaceutically acceptable excipient.

A third non-limiting aspect of the present disclosure provides use of the PYY₃₋₃₆ analog or the composition for treating a disease; or use of the PYY₃₋₃₆ analog or the composition in the preparation of a medicament for treating a disease; or a method for treating a disease, comprising administering an effective amount of the PYY₃₋₃₆ analog or the composition to a subject.

In some non-limiting embodiments, the present disclosure provides use of the PYY₃₋₃₆ analog or the composition for treating an NPYR-mediated disease; or use of the PYY₃₋₃₆ analog or the composition in the preparation of a medicament for treating an NPYR-mediated disease; or a method for treating an NPYR-mediated disease, comprising administering an effective amount of the PYY₃₋₃₆ analog or the composition to a subject.

In some non-limiting embodiments, the NPYR-mediated disease comprises obesity and related complications, diabetes and related complications, glucose intolerance and related diseases caused by glucose intolerance, hyperglycemia, hyperinsulinemia, hypertension, dyslipidemia, cognitive disorder, atherosclerosis, myocardial infarction, cardiovascular diseases, stroke, related diseases caused by intestinal permeability disorder such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, nephropathy, asthma, chronic obstructive pulmonary disease, dysmetabolic syndrome, mood disorders such as anxiety and depression, and neurodegenerative diseases such as Alzheimer's disease.

The present disclosure provides use of the PYY₃₋₃₆ analog or the composition in the preparation of a medicament for reducing food intake and increasing insulin sensitivity.

The present disclosure provides use of the PYY₃₋₃₆ analog or the composition in the preparation of a medicament for inhibiting fat accumulation.

In some non-limiting embodiments, the fat accumulation comprises subcutaneous fat accumulation, inter-organ fat accumulation, and liver fat accumulation.

The present disclosure provides use of the PYY₃₋₃₆ analog or the composition in the preparation of a health care product, a nutritional supplement and as a food additive ingredient.

Unless otherwise indicated, the amounts of different ingredients and reaction conditions as used herein should be interpreted as “about” and “approximate” in any case. Accordingly, unless otherwise specified, the numerical parameters stated in the following and appended claims are all general parameters, and different numerical parameters may be obtained due to different standard deviations under respective experimental conditions.

### Beneficial Effects

The present disclosure discloses a novel human PYY₃₋₃₆ analog. Compared with the naturally-occurring sequence of PYY₃₋₃₆, it has a remarkable effect on ameliorating diseases such as obesity, diabetes and fatty liver, and on mood disorders such as anxiety and depression.

### BRIEF DESCRIPTION OF THE DRAWINGS

The terms Fig., Figs., Figure, and Figures are used interchangeably in the specification to refer to the corresponding figures in the drawings.

FIG. 1 illustrates the effect of the polypeptide of Formula II (denoted by P1 in the figure, the same as follows) on cytotoxicity to HEK-293t cells by CCK-8 assay;

FIG. 2 illustrates the effect of the polypeptide of Formula II on cAMP content by cyclic AMP kit assay;

FIGS. 3A-3B illustrate the effect of the polypeptide of Formula II on body weight (FIG. 3A) and food intake (FIG. 3B) of HFD mice;

FIGS. 4A-4D illustrate the effect of the polypeptide of Formula II on anxiety and depression in mice, wherein FIG. 4A shows the residence time in a central area in an open field test, FIG. 4B shows the number of times of access to the central area in the open field test, FIG. 4C shows the residence time in an open arm area in an elevated plus maze test, and FIG. 4D shows the time of immobility in a tail suspension test;

FIGS. 5A-5B illustrates the effect of the polypeptide of Formula II on blood glucose in HFD mice by GTT (FIG. 5A) and ITT (FIG. 5B) tests;

FIGS. 6A-6B illustrates the effect of the polypeptide of Formula II on fatty liver by an oil red O staining test, wherein FIG. 6A shows the oil red O staining of the liver section and FIG. 6B shows the quantification of oil red O staining of the liver section;

FIGS. 7A-7B illustrates the effect of the polypeptide of Formula II on muscle by HE staining, wherein FIG. 7A shows HE staining of the muscle section and FIG. 7B shows quantification of HE staining of the muscle section; and

FIG. 8 illustrates a grouping design diagram of mice of Example 5.

### DETAILED DESCRIPTION

The following description sets forth exemplary embodiments of the present disclosure. It should be recognized that such a description is not intended to be a limitation on the scope of the present disclosure but is provided as a description of exemplary embodiments.

As used herein, the term “PYY₃₋₃₆ analog” refers to a human peptide tyrosine tyrosine (PYY) analog or a variant thereof, and is used to refer to polypeptides represented by Formulas I and II. The “PYY₃₋₃₆ analog” possesses PYY₃₋₃₆ activity. Possessing PYY₃₋₃₆ activity refers to the ability to bind PYY₃₋₃₆ receptor and trigger signal transduction pathway to produce insulin-promoting effect or other physiological effects.

As used herein, the term “amino acid” includes compounds as represented by the following general structures:

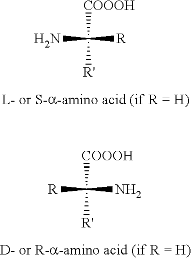

Wherein R and R′, unless otherwise specified, and as discussed herein, are used alone or as part of another moiety, and the term “amino acid” comprises an amino group and a carboxyl group which are attached to the same carbon known as “α” carbon, wherein R and/or R′ can be natural or unnatural side chains, including hydrogen. The absolute “S” configuration at the “α” carbon is usually known as “L” or “natural configuration”. In the case where both the “R” and the “R substituent” are hydrogen, the amino acid is glycine and is not chiral. Unless otherwise specified, the term “amino acid”, as herein used alone or as part of another moiety includes, but is not limited to, natural or unnatural amino acids, and wherein the carboxyl group is substituted, for example, valine, glycine, alanine, arylalanine, and heteroaryl alanine.

The PYY₃₋₃₆ analog of the present disclosure can be prepared by chemical synthesis using various solid-phase technologies^{[14-16]}.

The strategy used in the present disclosure is based on the Fmoc (9-fluorenylmethyloxycarbonyl) group for the temporary protection of the α-amino group, in combination with a tert-butyl group for the temporary protection of the amino acid side chain^{[14, 15, 17]}.

The PYY₃₋₃₆ analog of the present disclosure can be synthesized stepwise from the C-terminal of the peptide on an insoluble polymer carrier (also known as “resin”). The synthesis begins by attaching the C-terminal amino acid of the peptide to the resin by forming an amide or ester bond. This leads to the final release of the obtained peptide in the form of a C-terminal amide or carboxylic acid, respectively. Alternatively, if a C-terminal amino acid is present, the C-terminal residue as described herein may be attached to a 2-methoxy-4-alkoxybenzyl alcohol resin, and after the peptide sequence is assembled, the resulting peptide is released using a solution of LiBH₄ in THF^{[18]}.

It is known to those skilled in the art that an amino acid exists as two isomers, D isomer and/or L isomer. The present disclosure comprises a mixture of one isomer and/or the other isomer of an amino acid, which is used for the synthesis of the peptide as described herein.

The peptide of the PYY₃₋₃₆ analog of the present disclosure can be produced by recombination method, i.e., by culturing a host cell comprising a DNA sequence encoding the analog and being capable of expressing the peptide in a suitable nutrient medium under a condition that allow for the expression of the peptide. Non-limiting examples of host cells suitable for expressing such peptides are Escherichia coli, Saccharomyces cerevisiae, and mammalian BHK or CHO cell lines. In some non-limiting embodiments, this complete recombination fermentation step of the production process meets requirement, for example, for the sake of economical production. An inclusion body of a fusion protein comprising a PYY₃₋₃₆ analog backbone is subjected to denaturation and renaturation to obtain a fusion protein with a correct conformation. After a series of treatments such as digestion, sedimentation by adjustment, and centrifugation, the PYY₃₋₃₆ analog backbone with a higher content is obtained. After purification treatment by ion exchange chromatography, the PYY₃₋₃₆ analog backbone with a higher purity is obtained.

The term “excipient” in a broad sense refers to any ingredient in addition to the active therapeutic ingredient. The excipient may be a pharmaceutically acceptable inert substance, inactive substance and/or non-pharmaceutically active substance. Formulation of a pharmaceutically active ingredient with various excipients is known in the art^{[19]}. Non-limiting functional examples of excipients include, but are not limited to, a filler, a binder, a lubricant, a solvent, a disintegrant, a buffer, a preservative, etc. Those skilled in the art can easily select an excipient with a suitable function according to the dosage form of the preparation. For suitable carriers, reference can be made to^{[20]}.

The term “filler” includes, but is not limited to, lactose, sucrose, etc.; the term “binder” includes, but is not limited to, starch, gelatin, etc.; the term “lubricant” includes, but is not limited to, magnesium stearate, etc.; the term “preservative” includes, but is not limited to, sodium benzoate, etc.; the term “disintegrant” includes, but is not limited to, methylcellulose, dry starch agar, etc.; the term “buffer” includes, but is not limited to, sodium chloride, etc.; and the term “solvent” includes, but is not limited to, ethanol, water, etc.

The term “treating a disease” refers to treating and caring for a patient who has already been afflicted with a disease, condition, or disorder. The purpose of the treatment is to resist the disease, condition, or disorder. The treatment includes administration of an active compound to eliminate or control a disease, condition, or disorder, and to alleviate symptoms or complications associated with the disease, condition, or disorder.

The term “NPYR-mediated disease” refers to an effect produced by binding NPYR receptor to activate the downstream pathway. Since NPYR receptor is widely expressed in the body, the NPYR-mediated disease includes, but is not limited to, obesity and related complications, diabetes and related complications, glucose intolerance and related diseases caused by glucose intolerance, hyperglycemia, hyperinsulinemia, hypertension, dyslipidemia, cognitive disorder, atherosclerosis, myocardial infarction, cardiovascular diseases, stroke, related diseases caused by intestinal permeability disorder such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, nephropathy, asthma, chronic obstructive pulmonary disease, dysmetabolic syndrome, mood disorders such as anxiety and depression, and neurodegenerative diseases such as Alzheimer's disease.

The scope of the present disclosure includes a PYY₃₋₃₆ analog and a composition comprising the PYY₃₋₃₆ analog, wherein the composition comprises the PYY₃₋₃₆ analog as an active ingredient and a pharmaceutically acceptable excipient that can be used in combination with PYY₃₋₃₆. The PYY₃₋₃₆ analog of the present disclosure is used alone or in combination with one or more therapeutic agents for other conditions, for treating related diseases, as well as their used in the preparation of a medicament for reducing food intake, reducing β cell apoptosis, increasing the function of islet β cells, increasing β-cell clusters and/or restoring sensitivity of glucose to β-cells.

The PYY₃₋₃₆ analog of the present disclosure can be pharmaceutically administered in a variety of preparation dosage forms (each dosage form includes dosage forms of immediate-release, sustained-release, or controlled-release), including but not limited to dosage forms such as tablet, capsule, injection. Those skilled in the art can choose a suitable preparation dosage form according to the route of administration. The route of administration includes, but is not limited to, oral administration, transdermal administration, subcutaneous administration, intravenous administration, intramuscular administration, etc. According to the route of administration, the dosage form is formulated on the basis of acceptable pharmaceutical operation procedures^{[21]}. All the dosage forms used are well known to relevant technicians in the pharmaceutical field, and they are administered after mixing with pharmaceutically acceptable excipient/excipients which is/are selected according to the selected route of administration and standard pharmaceutical operation procedures.

The PYY₃₋₃₆ analog or composition of the present disclosure may also be administered by coupling with a soluble polymer such as a carrier targeting a drug, cross-linking with a biodegradable polymer or hydrogel for controlling drug release, or combining with an amphiphilic block copolymer.

The dosage regimen of the preparation of the present disclosure will vary depending on known factors, such as the pharmacokinetic properties of a specific agent and the mode and route of administration thereof, the species, health status, medical status, age, sex and body weight of the recipient, the nature and severity of the symptoms, the type and frequency of treatment, the kidney and liver functions of the patient, as well as the desired effect. Physicians or veterinarians can formulate prescriptions according to the required amount of the drug that is effective in preventing, resisting, or inhibiting the progress of the disease state.

Administration of the therapeutic agent of the present disclosure includes, but is not limited to, administering a therapeutically effective amount of the agent of the present disclosure. The term “therapeutically effective amount” refers to the amount of a therapeutic agent for treating or preventing a condition that can be treated or prevented by administering the composition of the present disclosure. The dosage is an amount that is sufficient to show a therapeutic or preventive or ameliorative effect. The effect includes the treatment or prevention of the diseases described herein. The exact and effective amount for the subject will depend on the body weight and health status of the subject, the nature and severity of the condition to be treated, the advice given by the attending physician, and the therapeutic method or a combination of therapeutic methods selected for administration. Therefore, it is useless to specify the exact and effective amount in advance.

According to the general guideline, in the composition, the active ingredient will generally be present in an amount of about 0.5-95% based on the total weight of the composition. The composition of the present disclosure can be administered in a single daily dose or in a total daily dose divided into two, three or four doses per day. The composition of the present disclosure may also be administered as a long-acting preparation, which will enable the active ingredient to be slowly released within a period of days/weeks/months as required.

### Abbreviations

| Abbreviation | Full name | Abbreviation | Full name | Abbreviation | Full name | Abbreviation | Full name |
|---|---|---|---|---|---|---|---|
| Fmoc | 9-fluorenylmethyl- | Ile | Isoleucine | g | Gram | PYY | Peptide |
| | oxycarbonyl | | | | | | tyrosine |
| | | | | | | | tyrosine |
| DMF | Dimethylformamide | Leu | Leucine | mg | Milligram | DM | Diabetes |
| | | | | | | | mellitus |
| NMM | N-methylmorpholine | Lys | Lysine | μg | Microgram | h | Hour |
| DCM | Dichloromethane | Met | Methionine | μmol | Micromole | AFLD | Alcoholic |
| | | | | or | | | fatty liver |
| | | | | μM | | | |
| LiBH4 | Lithium | Phe | Phenylalanine | NS | Normal | T2DM | Type 2 |
| | borohydride | | | | | | diabetes |
| | | | | | | | mellitus |
| HBTU | O-benzotriazole- | Pro | Proline | nm | Nanometer | EPM | Elevated plus |
| | tetramethyluronium | | | | | | maze test |
| | hexafluorophosphate | | | | | | |
| HCl | Hydrogen chloride | Ser | Serine | mL or ml | Milliliter | HFD | High-fat diet |
| NaOH | Sodium hydroxide | Thr | Threonine | AMP | Adenosine | ITT | Insulin |
| | | | | | monophosphate | | tolerance |
| | | | | | | | test |
| Ala | Alanine | Trp | Tryptophan | Kg | Kilogram | GTT | Glucose |
| | | | | | | | tolerance |
| | | | | | | | test |
| Arg | Arginine | Tyr | Tyrosine | OFT | Open field test | U/Kg | Unit/kilogram |
| Asn | Asparagine | Val | Valine | IC50 | Half-inhibitory | FL | Fatty liver |
| | | | | | concentration | | |
| Asp | Aspartic acid | Dhval | 2,3-didehydrovaline | BHK | Baby hamster | CHO | China hamster |
| | | | | | kidney | | ovary |
| Cys | Cysteine | Dhthr | Dehydroxythreonine | ° C. | degree Celsius | NAFLD | Non-alcoholic |
| | | | | | | | fatty liver |
| Gln | Glutamine | Dah | 2,7-diaminoheptanoic | DPP- | Enzyme dipeptidyl | cAMP | Cyclic adenosine |
| | | | acid | IV | peptidase IV | | monophosphate |
| Glu | Glutamic acid | Orn | Ornithine | Dap | 2,3-diaminopropionic | His | Histidine |
| | | | | | acid | | |
| Gly | Glycine | Dhser | Dehydroxyserine | NPY2R | Neuropeptide Y2 | min | Minute |
| | | | | | receptor | | |
| CO₂ | Carbon dioxide | DNA | Deoxyribonucleotide | D- | Dextrorotatory/ | HE | Hematoxylin- |
| | | | | and/or | levorotatory | | eosin |
| | | | | L- | | | |
| TST | Tail suspension | pH | Power of | / | / | / | / |
| | test | | hydrogen | | | | |

### Example 1. General Method for Chemical Synthesis of Polypeptide of Formula I

Formula I
IKP EAP GEX₁₁ ASP EEL X₁₈X₁₉Y YAX₂₃ LRH X₂₇LN
X₃₀VT RQRY

Without changing the secondary structure of Formula I, amino acid substitution at a specific site was realized according to the principle of conservative substitution of amino acids, wherein:
X₁₁ was selected from the group consisting of aspartic acid, glycine, glutamic acid, and asparagine;
X₁₈ was selected from the group consisting of aspartic acid, glutamine, and histidine;
X₁₉ was selected from the group consisting of arginine and lysine;
X₂₃ was selected from the group consisting of serine, threonine, alanine, glycine, and methionine;
X₂₇ was selected from the group consisting of tyrosine, tryptophan, and phenylalanine; and
X₃₀ was selected from the group consisting of leucine, alanine, isoleucine, valine, and methionine.

Formula I of the present disclosure was synthesized stepwise from the C-terminal of the peptide on an insoluble polymer carrier (also known as “resin”). The synthesis began by attaching the C-terminal amino acid of the peptide to the resin by forming an amide or ester bond. This led to the final release of the obtained peptide in the form of a C-terminal amide or carboxylic acid, respectively. Alternatively, when a C-terminal amino acid was present, the C-terminal residue as described herein was attached to a 2-methoxy-4-alkoxybenzyl alcohol resin (SASRIN™, Bachem Bioscience, Inc., King of Prussia, PA), and after the peptide sequence was assembled, the resulting peptide was released using a solution of LiBH₄ in THF^{[18]}.

### Example 2. General Method for Chemical Synthesis of Polypeptide of Formula II (P1)

a. Resin swelling: Fmoc-Ala-Wang resin was poured into a reaction column, DCM was added and soaked for 30 minutes, and drained.
b. Deprotection: an appropriate amount of a deprotection solution was added to the reaction column, nitrogen was introduced, and the solution was stirred for 30 minutes and drained.
c. Weighing: a protected amino acid in an molar amount of 3 times that of the resin was measured, and benzotriazole-N,N,N,N-tetramethyluronium hexafluorophosphate (HBTU) in an molar amount of 3 times was weighed.
d. Washing for deprotection: an appropriate amount of DMF was added to the reaction column, sparged with nitrogen for 2 minutes, and drained. The operation was repeated for 6 times.
e. Charging: the prepared protected amino acid and HBTU were added to the reaction column, and NMM in a molar amount of 6 times that of the resin was added and stirred with nitrogen for 30 minutes.
f. Washing after reaction: the resulting solution was drained from the reaction column, an appropriate amount of DMF was added for washing, sparged with nitrogen for 2 minutes, and drained. The operation was repeated for 3 times.
g. Detection: an appropriate amount (10-20 pieces) of the resin was taken into a small test tube, and two drops of each of Solutions A, B, and C were added, and the resulting material was put into a dry heater and heated for 3 minutes (110° C.) for a reaction. When the solution was yellowish and the resin was colorless and transparent, the reaction was complete, and the next amino acid could be connected. The above steps b-f were repeated until the last amino acid was connected. (Solution A: 80% phenol+20% anhydrous ethanol; Solution B: redistilled pyridine; Solution C: 5 g ninhydrin+100 mL anhydrous ethanol)
h. Washing and drying after synthesis: the last amino acid was connected, deprotected, and washed. After draining, an appropriate amount of methanol was added to the reaction column, sparged with nitrogen for 2 minutes and drained; and an appropriate amount of DCM was then added, sparged with nitrogen for 2 minutes and drained. The operation was repeated for 3 times. An appropriate amount of methanol was added to a reaction kettle, sparged by nitrogen for 2 minutes and drained. The operation was repeated twice. The resin was put into a vessel and vacuum-dried in a vacuum dryer for 12 hours.
i. Releasing of polypeptide: a centrifuge tube was sealed, put into a centrifuge, and centrifuged for 3 minutes at 4000 rpm/min; supernatant was removed, ethyl ether was added, and the resulting mixture was stirred evenly and centrifuged again. The operation was repeated for 5 times. A resulting polypeptide was put into a vacuum dryer and dried for 24 hours to obtain a crude polypeptide product as a white powder and weighed.

### Example 3. Cytotoxicity of the Polypeptide of Formula II

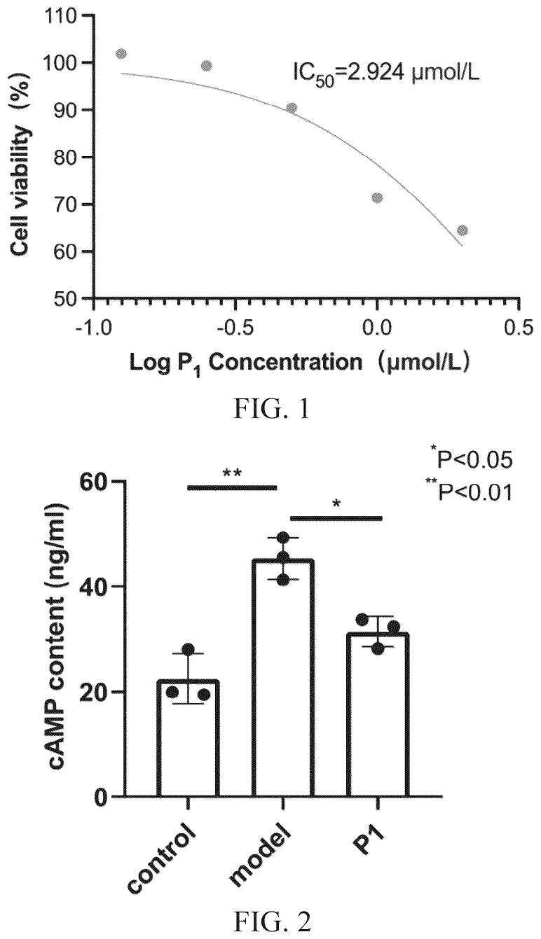

HEK-293t cells were cultured in a 96-well plate (37±0.5° C., 5±0.1% CO₂). 0.16 mg of the polypeptide of Formula II was dissolved in 10 ml of PBS (4 μmol/mL) and serial diluted according to a concentration gradient (2 μmol/mL, 1 μmol/mL, 0.5 μmol/mL, and 0.25 μmol/mL), and then a high-glucose DMEM complete medium (Wuhan Servicebio Technology Co., Ltd., the same as follows) was added to the respective dilutions. The toxicity was assayed by a CCK-8 kit (Wuhan Pricella Biotechnology Co., Ltd.), absorbance at 450 nm was measured by a microplate reader, cell survival rate was calculated, and an IC50 curve was plotted by GraphPad Prism (FIG. 1).

Based on the IC50 results of the polypeptide of Formula II, an administration dosage for animal was estimated according to the following formula:

Administration dosage for animal (mg/kg)=Administration dosage for cells (μg/mL)×liquid intake (mL/kg)÷animal body weight (kg).

After conversion, the administration dosage for animals was 2356 μM/kg. In an actual animal experiment, 80 μM/kg was used as the administration dosage (approximately 3% of 2356 μM/kg).

### Example 4 Determination of Cyclic AMP

HEK-293t cells were cultured in a 96-well plate and transfected with a recombinant expression plasmid pCAGIG of the polypeptide of Formula II by using Lipo2000 transfection reagent (Beijing Solarbio Science & Technology Co., Ltd.). After 24 hours of culturing, detection was carried out by a cAMP kit (Beijing Solarbio Science & Technology Co., Ltd.), absorbance at 450 nm was measured by a microplate reader and analyzed by GraphPad Prism.

The results showed that (as shown in FIG. 2) compared with the model group, the cAMP content of the group of the polypeptide of Formula II was decreased by about 5-40%, indicating that the polypeptide of Formula II could reduce the level of cAMP in cells by activating Gi/o protein and inhibiting adenylate cyclase. This was consistent with the regulation result of endogenous PYY₃₋₃₆. Therefore, the polypeptide of Formula II possessed the activity of endogenous PYY₃₋₃₆.

### Example 5. Effect of the Polypeptide of Formula II on Body Weight

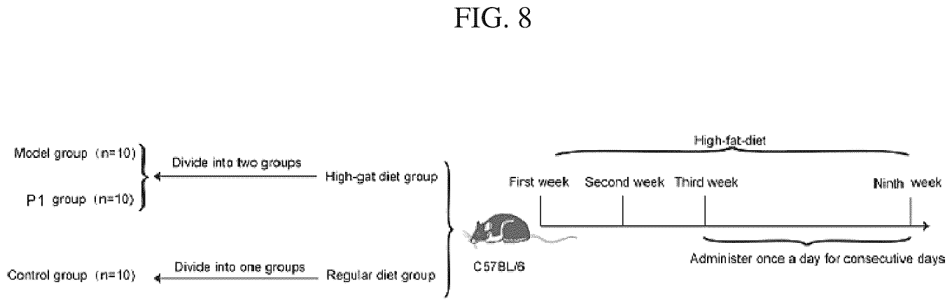

30 male C57BL/6 SPF mice (Sipeifu (Beijing) Biotechnology Co., Ltd., the same as follows) were raised in an environment at a temperature of 22-26° C. and a relative humidity of 40-70%. The mice were divided into three groups (10 mice per group, as shown in FIG. 8): normal control group (regular diet and normal saline, NS), HFD model group (high-fat diet and normal saline), and a group administered with the polypeptide of Formula II (high-fat diet and the polypeptide of Formula II, P1). On days 1-7, 50% high-fat diet was fed for adaptive diet induction, and on days 8-50, 100% high-fat diet was fed. On days 15-50, 0.9% normal saline or the polypeptide of Formula II was daily injected subcutaneously, and the daily body weight change and the total food intake 48 h after each administration were measured.

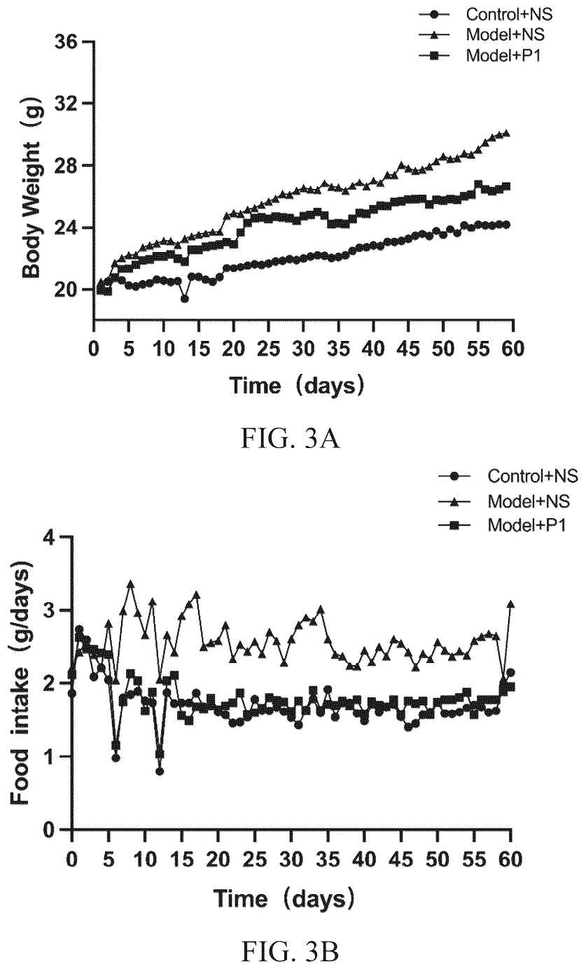

The results for body weight were as shown in FIG. 3A. From day 5, the body weight difference among the three groups of mice began to change obviously. The control group was fed with regular diet, and the body weight increased slowly, with a body weight difference of about 5-30% from that of the model group and about 5-15% from that of the group of the polypeptide of Formula II. Where the model group and the group of the polypeptide of Formula II were both fed with high-fat diet, the body weight difference of the model group was increased by about 5-30% more than that of the group of the polypeptide of Formula II.

The results for food intake were as shown in FIG. 3B. From days 6-60, the food intake of the model group increased by about 5-65% compared with the normal control group, and the food intake of the group administered with the polypeptide of Formula II decreased by 10-60% compared with the model group.

### Example 6. Anti-Anxiety and Anti-Depression Effects of the Polypeptide of Formula II

The three groups of mice in Example 5 were subjected to a behavioral experiment. Open field test (OFT) (Pullman): the mice were put into a test box to detect their activities during the test period. Elevated plus maze (EPM) (Pullman): the mice were placed in the center of the elevated plus maze to observe their activities during the test period. Tail suspension test (TST): the tails of the mice were hung on a cage rack, and the behavior patterns of struggle, swing and immobility were observed during the suspension.

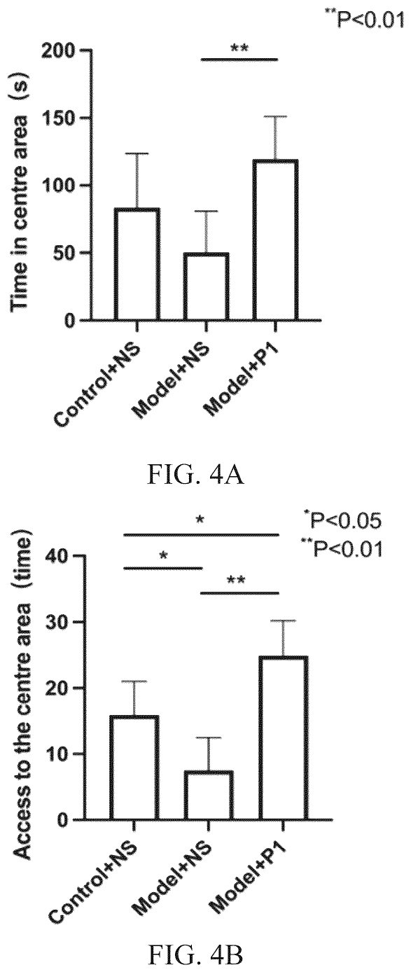

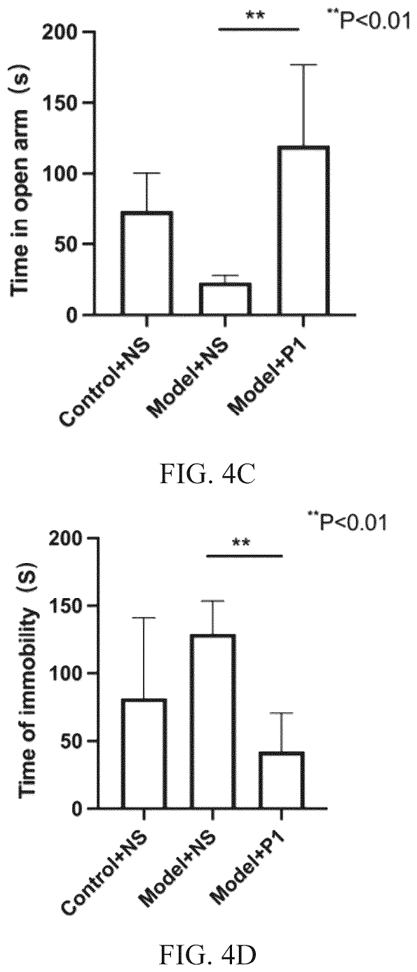

In the OFT and EPM experiments (as shown in FIGS. 4A-4C), compared with the control group, the movement time in the central area and the number of access to the central area of the model group were both significantly reduced, and the movement time in the open arm area was reduced. While compared with the model group, the movement time in the central area and the number of access to the central area of the group administered with the polypeptide of Formula II were both significantly increased, and the movement time in the open arm area was also significantly increased. In the TST experiment (as shown in FIG. 4D), the immobility time of the model group was increased compared with that of the control group, and the immobility time of the group administered with the polypeptide of Formula II was significantly decreased compared with the model group and control group.

The above results showed that obesity could aggravate the anxiety and depression symptoms of the mice, and the administration of the polypeptide drug of Formula II to the mice with anxiety and depression symptoms could significantly ameliorate their anxiety and depression symptoms.

### Example 7. Effect of the Polypeptide of Formula II on Blood Glucose

The mice of Example 5 were fasted for one day, during which they drank water normally. After administrating 20% glucose by gavage, the blood glucose level was determined with blood glucose meter and blood glucose test paper at 15 min, 30 min, 60 min, 90 min and 120 min.

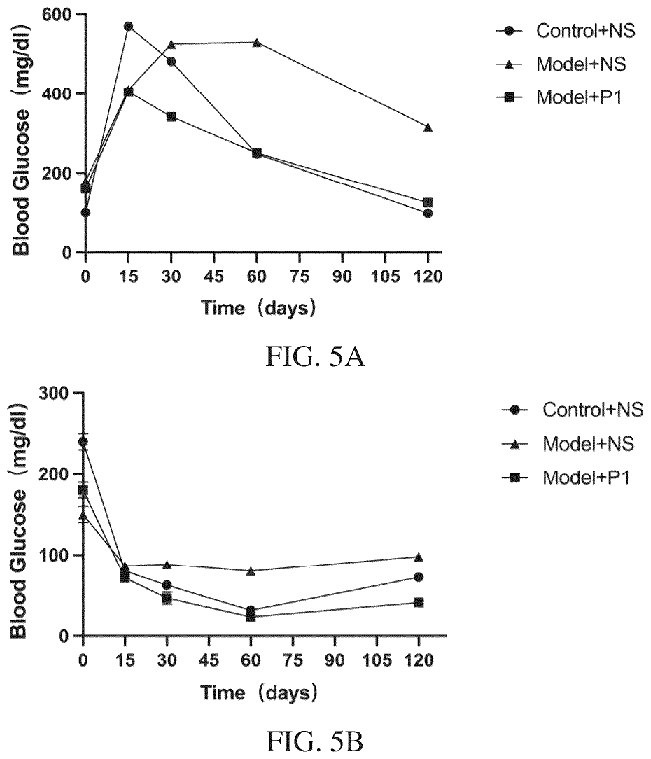

The GTT results were as shown in FIG. 5A. The blood glucose of the mice of the model group increased significantly by 5-55% within 15-120 min compared with the control group; and compared with the model group, the blood glucose of the group administered with the polypeptide of Formula II decreased by 5-55% within 15-120 min, indicating that the polypeptide of Formula II had an obvious effect on lowering blood glucose.

### Example 8. Effect of the Polypeptide of Formula II on Insulin Tolerance

The mice of Example 5 were fasted for one day, during which they drank water normally. After intraperitoneal injection of 0.75 U/kg insulin solution, the blood glucose level was determined with blood glucose meter and blood glucose test paper at 15 min, 30 min, 60 min, 90 min and 120 min.

The ITT results were as shown in FIG. 5B. Within 120 min, the blood glucose of the control group decreased by 5-90%, that of the model group decreased by 5-50%, and that of the group administered with the polypeptide of Formula II decreased by 10-75%. Compared with the model group, the group administered with the polypeptide of Formula II had improved insulin tolerance.

### Example 9. Effect of the Polypeptide of Formula II on Fat Accumulation in the Body

The mice of Example 5 were anesthetized and then fixed on a mouse plate supinely, the outer skin was dissected to expose subcutaneous fat, and the subcutaneous fat accumulation was recorded. Organs were subsequently exposed, and the inter-organ fat accumulation was observed. Finally, muscle and liver tissues were collected from the mice and stored in a refrigerator at −80° C.

Compared with the control group, the subcutaneous and inter-organ fat accumulation in the model group increased by 40-50%, and administration of the polypeptide of Formula II resulted in an increase of 20-30% of fat accumulation compared with the control group and a decrease of 5-20% compared with the model group, indicating that the group administered with the polypeptide of Formula II had an obvious control effect on reducing subcutaneous and inter-organ fat accumulation.

### Example 10. Effect of the Polypeptide of Formula II on Fatty Liver

The mouse liver tissue collected in Example 9 was frozen and sectioned. The liver tissue sections were fixed with formaldehyde-calcium, then washed, and immersed with 60% isopropanol. A saturated oil red O stock solution (Beijing Solarbio Science & Technology Co., Ltd.) was diluted at ratio of 3:2 (oil red O: distilled water) for staining, and the sections were mounted with glycerol gelatin and examined under a microscope.

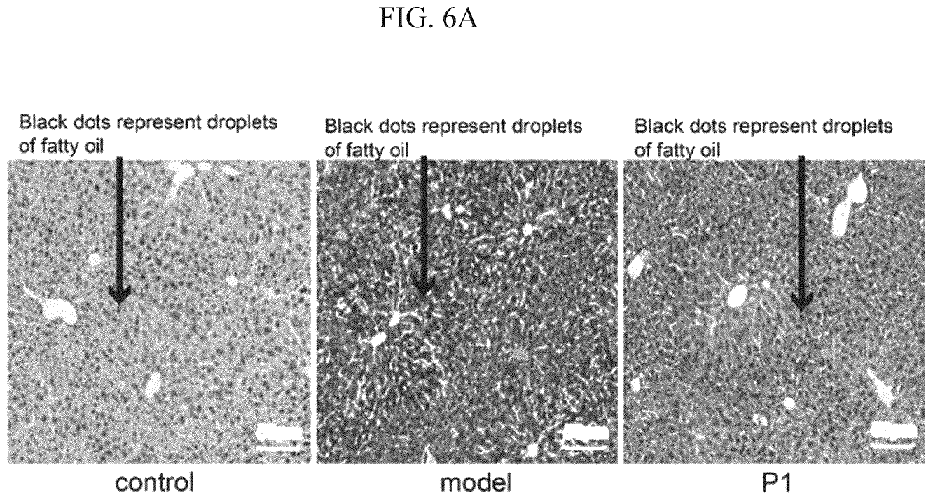

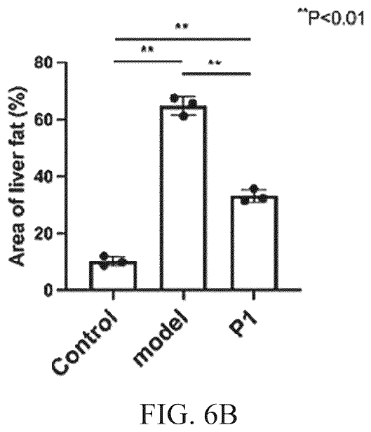

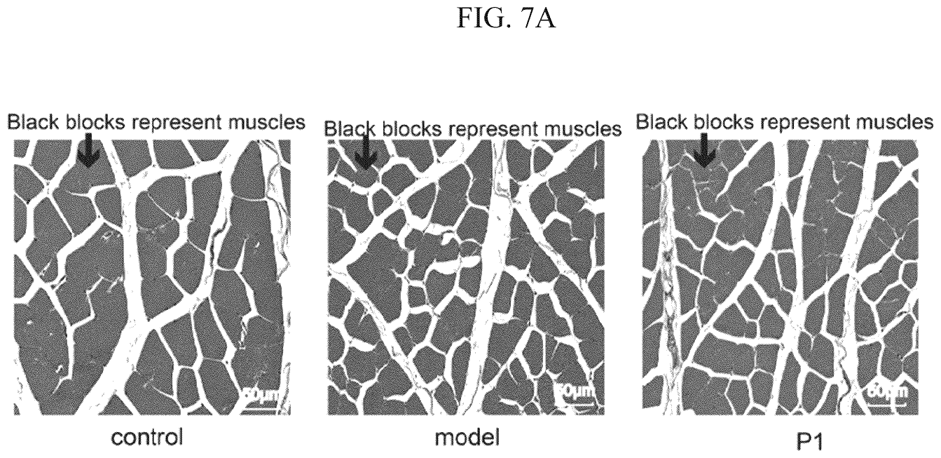

The results were as shown in FIGS. 6A-6B. Compared with the control, fatty oil droplets of the model group increased by 40-60%, and compared with the model group, fatty oil droplets of the group administered with the polypeptide of Formula II decreased by 20-40%, indicating that the polypeptide of Formula II had a significant effect on reducing liver fat accumulation.

### Example 11. Effect of the Polypeptide of Formula II on Muscle

The mouse muscle tissue collected in Example 9 was frozen and sectioned. After staining with a hematoxylin solution and an eosin solution (Beijing Solarbio Science & Technology Co., Ltd.), the sections were mounted with glycerol gelatin and examined under a microscope.

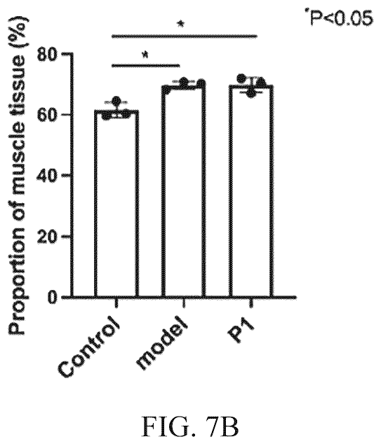

The results were as shown in FIGS. 7A-7B. The muscle area of the model group increased by 3-15% compared with the control group, and the muscle area of the group administered with the polypeptide of Formula II was not significantly different from that of the model group, indicating that continuous administration of the polypeptide of Formula II had no obvious effect on the muscle content in mice.

### Example 12. Preparation of Pharmaceutical Preparations of the Polypeptide of Formula II

### A) Tablets

Tablets could be prepared according to conventional preparation operating procedures for tablets, whereby tablets with preparation specifications were obtained, which were made up of, for example, 100 g of the polypeptide of Formula II, 0.2 g of colloidal silicon dioxide, 5 g of magnesium stearate, 275 g of microcrystalline cellulose, 11 g of starch, and 98.8 g of lactose. An appropriate coating could be used to improve the taste or delay absorption. 1000 tablets were prepared.

### B) Capsules

Capsules could be prepared according to conventional preparation operating procedures for capsules, whereby capsules with preparation specifications were obtained. For example, 1000 capsules were prepared by filling standard hard gelatin capsules with 100 g of the polypeptide of Formula II, 150 g of lactose, 50 g of cellulose, 6 g of magnesium stearate, and an appropriate amount of an absorbent promoter.

### C) Injections

An injection could be prepared by conventional procedures. For example, an injection preparation with the following composition was prepared by the method described below.

| Component | Amount |
|---|---|
| Polypeptide of Formula II | 10 g |
| HCl or NaOH | To adjust the pH value to 6-8 |
| SBE-cyclodextrin (Captisol) | 55 g |
| Water for injection | Added to reach 5 L |
| Aqueous solution of sodium chloride | Appropriate amount |

The weighed polypeptide of Formula II was dissolved in part of water for injection with optimal pH value, and SBE-cyclodextrin was added to the drug solution and stirred for about 8 min. NaOH or HCl was added to adjust the pH value to a desired value (between 6 and 8). Water for injection and an aqueous solution of sodium chloride were added respectively to achieve a final volume of 5 L and to make the solution isotonic. The solution was then divided and filled into appropriate ampoules to prepare 1000 vials of injections. Before adjusting the pH value, other inactive components such as solvents, cosolvents, solubilizers, antioxidants, reducing agents, preservatives, buffers, protectants, tension regulators and other special additives could be added as required. The injection preparation was necessarily sterile, pyrogen-free, and free of particulate matters when it was a solution. It could be prepared by stirring in a pharmaceutically acceptable buffer with or without a cosolvent or other excipients. Before use, the solution should be made isotonic with pharmaceutical sodium chloride and sterilized.

Certain embodiments or aspects of the present disclosure have been described above with reference to the accompanying drawings and are not thus intended to limit the scope of right of the present disclosure. Any modifications, equivalent substitutions and improvements made by those skilled in the art without departing from the scope and essence of the present disclosure shall all fall within the scope of right of the present disclosure.

### REFERENCE

ADDIN EN.REFLIST [1] Pierre J F, Peters B M, La Torre D, et al. Peptide YY: A Paneth cell antimicrobial peptide that maintains Candida gut commensalism [J]. Science, 2023, 381(6657): 502-8.
[2] Batterham R L, Cowley M A, Small C J, et al. Gut hormone PYY(3-36) physiologically inhibits food intake [J]. Nature, 2002, 418(6898): 650-4.
[3] Guida C, Stephen S D, Watson M, et al. PYY plays a key role in the resolution of diabetes following bariatric surgery in humans [J]. EBioMedicine, 2019, 40: 67-76.
[4] Somero G N. Comparative physiology: a “crystal ball” for predicting consequences of global change [J]. American Journal of Physiology-Regulatory, Integrative and Comparative Physiology, 2011, 301(1): R1-R14.
[5] Bessesen D H. Update on Obesity [J]. The Journal of Clinical Endocrinology & Metabolism, 2008, 93(6): 2027-34.
[6] Bhaskaran K, Douglas I, Forbes H, et al. Body-mass index and risk of 22 specific cancers: a population-based cohort study of 5·24 million UK adults [J]. Lancet, 2014, 384(9945): 755-65.
[7] Singh G M, Danaei G, Farzadfar F, et al. The age-specific quantitative effects of metabolic risk factors on cardiovascular diseases and diabetes: a pooled analysis [J]. PLoS One, 2013, 8(7): e65174.
[8] Wormser D, Kaptoge S, Di Angelantonio E, et al. Separate and combined associations of body-mass index and abdominal adiposity with cardiovascular disease: collaborative analysis of 58 prospective studies [J]. Lancet, 2011, 377(9771): 1085-95.
[9] Alford S, Patel D, Perakakis N, et al. Obesity as a risk factor for Alzheimer's disease: weighing the evidence [J]. Obes Rev, 2018, 19(2): 269-80.
[10] Safaei M, Sundararajan E A, Driss M, et al. A systematic literature review on obesity: Understanding the causes & consequences of obesity and reviewing various machine learning approaches used to predict obesity [J]. Computers in Biology and Medicine, 2021, 136: 104754.
[11] Kim C, Costello F J, Lee K C, et al. Predicting Factors Affecting Adolescent Obesity Using General Bayesian Network and What-If Analysis [J]. Int J Environ Res Public Health, 2019, 16(23).
[12] Stefan N, Schick F, Birkenfeld A L, et al. The role of hepatokines in NAFLD [J]. Cell Metab, 2023, 35(2): 236-52.
[13] Shiha G, Korenjak M, Eskridge W, et al. Redefining fatty liver disease: an international patient perspective [J]. The lancet Gastroenterology & hepatology, 2021, 6(1): 73-9.
[14] Gross E. The Peptides Analysis, Synthesis, Biology: Modern Techniques of Conformational Structural, and Configurational Analysis [M]. Elsevier, 2012.
[15] Schröder E, Lübke K. The Peptides: Methods of peptide synthesis [M]. Elsevier, 2014.
[16] Chan W, White P. Fmoc solid phase peptide synthesis: a practical approach [M]. OUP Oxford, 1999.
[17] Atherton E, Sheppard R. The fluorenylmethoxycarbonyl amino protecting group [J]. ChemInform, 1988, 19(33): no-no.
[18] Bravis V, Kaur A, Walkey H C, et al. Relationship between islet autoantibody status and the clinical characteristics of children and adults with incident type 1 diabetes in a UK cohort [J]. BMJ Open, 2018, 8(4): e020904.
[19] Allen L V, Jr. Remington: The Science and Practice of Pharmacy: from the past into the future [J]. Int J Pharm Compd, 2012, 16(5): 358-62.
[20] Fox L M. The science and practice of pharmacy [J]. American Journal of Pharmaceutical Education, 2006, 70(3).
[21] Goodman L S. Goodman and Gilman's the pharmacological basis of therapeutics [M]. McGraw-Hill New York, 1996.

What is claimed is:

1. A PYY₃₋₃₆ analog comprising an amino acid sequence represented by Formula I below:
Formula I
(SEQ ID NO: 2)
IKP EAP GEX₁₁ ASP EEL X₁₈X₁₉Y YAX₂₃ LRH X₂₇LN
X₃₀VT RQRY
wherein:
X₁₁ is selected from the group consisting of aspartic acid, glycine, glutamic acid, and asparagine;
X₁₈ is selected from the group consisting of aspartic acid, glutamine, and histidine;
X₁₉ is selected from the group consisting of arginine and lysine;
X₂₃ is selected from the group consisting of serine, threonine, alanine, glycine, and methionine;
X₂₇ is selected from the group consisting of tyrosine, tryptophan, and phenylalanine; and
X₃₀ is selected from the group consisting of leucine, alanine, isoleucine, valine, and methionine.

2. A PYY₃₋₃₆ analog comprising a polypeptide sequence represented by Formula II below:
Formula II
(SEQ ID NO: 1)
IKP EAP GED ASP EEL VRY YAG LRH WLN LVT RQRY.

3. A composition comprising the PYY₃₋₃₆ analog according to claim 2, and a pharmaceutically acceptable excipient.

4. A method for treating an NPYR-mediated disease, comprising administering a therapeutically effective amount of the PYY₃₋₃₆ analog according to claim 2 to a subject in need thereof.

5. The method according to claim 4, wherein the NPYR-mediated disease comprises obesity and related complications, diabetes and related complications, glucose intolerance and related diseases caused by glucose intolerance, hyperglycemia, hyperinsulinemia, hypertension, dyslipidemia, cognitive disorder, atherosclerosis, myocardial infarction, cardiovascular diseases, stroke, related diseases caused by intestinal permeability disorder, apoplexy and/or hemorrhagic apoplexy, neuroinflammation, rheumatic or rheumatoid arthritis, nephropathy, asthma, chronic obstructive pulmonary disease, dysmetabolic syndrome, mood disorders, and neurodegenerative diseases.

6. A method for treating an NPYR-mediated disease, comprising administering a therapeutically effective amount of the composition according to claim 3 to a subject in need thereof.

7. The method according to claim 6, wherein the NPYR-mediated disease comprises obesity and related complications, diabetes and related complications, glucose intolerance and related diseases caused by glucose intolerance, hyperglycemia, hyperinsulinemia, hypertension, dyslipidemia, cognitive disorder, atherosclerosis, myocardial infarction, cardiovascular diseases, stroke, related diseases caused by intestinal permeability disorder, apoplexy and/or hemorrhagic apoplexy, neuroinflammation, rheumatic or rheumatoid arthritis, nephropathy, asthma, chronic obstructive pulmonary disease, dysmetabolic syndrome, mood disorders, and neurodegenerative diseases.

8. A method for reducing food intake, comprising administering a therapeutically effective amount of the PYY₃₋₃₆ analog according to claim 2 to a subject in need thereof.

9. A method for increasing insulin sensitivity, comprising administering a therapeutically effective amount of the PYY₃₋₃₆ analog according to claim 2 to a subject in need thereof.

10. A method for inhibiting fat accumulation, comprising administering a therapeutically effective amount of the PYY₃₋₃₆ analog according to claim 2 to a subject in need thereof.

11. The method according to claim 10, wherein the fat accumulation comprises subcutaneous fat accumulation, inter-organ fat accumulation, and liver fat accumulation.

12. A health care product comprising the PYY₃₋₃₆ analog according to claim 2.

13. A nutritional supplement comprising the PYY₃₋₃₆ analog according to claim 2.

14. A food additive comprising the PYY₃₋₃₆ analog according to claim 2.

15. A method for reducing food intake, comprising administering a therapeutically effective amount of the composition according to claim 3 to a subject in need thereof.

16. A method for increasing insulin sensitivity, comprising administering a therapeutically effective amount of the composition according to claim 3 to a subject in need thereof.

17. A method for inhibiting fat accumulation, comprising administering a therapeutically effective amount of the composition according to claim 3 to a subject in need thereof.

18. The method according to claim 17, wherein the fat accumulation comprises subcutaneous fat accumulation, inter-organ fat accumulation, and liver fat accumulation.

19. A health care product comprising the composition according to claim 3.

20. A food additive comprising the composition according to claim 3.
